(19) United States
(12) Patent Application Publication
(10) Pub. No.: US 2011/0146005 A1
(43) Pub. Date: Jun. 23, 2011

(54) CATIONIC DIHYDROISOQUINOLINIUM DERIVATIVES FOR USE AS BLEACH ACTIVATORS

(76) Inventors: Wibke Gross, Hueckelhoven (DE); Denise Fuhr, Hamburg (DE); Ralph Nemitz, Juechen (DE); Astrid Kleen, Hamburg (DE); Dorota Sendor-Mueller, Duesseldorf (DE)

(21) Appl. No.: 13/036,034

(22) Filed: Feb. 28, 2011

Related U.S. Application Data
(63) Continuation-in-part of application No. PCT/EP09/57012, filed on Jun. 8, 2009.

(30) Foreign Application Priority Data
Aug. 28, 2008 (DE) 102008044714.5

(51) Int. Cl. A61K 8/49; A61Q 5/10; A61Q 5/08

(52) U.S. Cl. 8/406

(57) ABSTRACT

Agent for dyeing while simultaneously bleaching keratin fibers. The agent contains, in a cosmetic carrier, at least one oxidation dye precursor and at least one cationic 3,4-dihydorisoquinolinium derivative.

### CROSS REFERENCE TO RELATED APPLICATIONS

The present application is a continuation of International Patent Application No. PCT/EP2009/057012 filed 8 Jun. 2009, which claims priority to German Patent Application No. 10 2008 044 714.5 filed 28 Aug. 2008, both of which are incorporated herein by reference.

The present invention relates to an agent for changing the color of and simultaneously lightening keratinous fibers, particularly human hair, containing, in a cosmetic carrier, at least one color-changing compound and a cationic derivative of 3,4-dihydroisoquinoline. The invention also relates to a method for applying such agents to keratinous fibers. The invention furthermore relates to use of the agent for dyeing and simultaneously lightening the hair.

Changing the shape and particularly the color of hair is an important area of modem cosmetics. It allows the appearance of hair to be adapted both to the latest fashion trends and the personal preferences of the individual. In order to change the color of hairstyles in line with fashion trends or to conceal gray or even white hair with fashionable or natural shades, the consumer turns to color-changing agents. In addition to having the desired coloring capacity, these agents should give rise to the minimum possible damage to the hair and should preferably even have additional conditioning properties.

One skilled in the art is familiar with various dye systems for providing color-changing cosmetic agents, particularly for skin or keratinous fibers such as human hair, depending on the requirements of the coloring process. For permanent, intense colors with corresponding fastness properties, oxidation coloring agents are used. For temporary colors, coloring or tinting agents are conventionally used which contain direct dyes as the coloring component. These are dye molecules which attach directly to the substrate and require no oxidative process to develop the color. Coloring the hair, particularly lightening and coloring dark hair, is a central desire of many consumers. For consumers with relatively strongly pigmented hair, achieving intense shades in the fashion and natural color range which are lighter than the original natural hair color requires adequate lightening. It is difficult to achieve significant lightening of dark blonde, brown or black hair by use of hydrogen peroxide alone.

Use of a combination of hydrogen peroxide and persulfate salts to intensify the lightening effect is known in the art. However, these mixtures of oxidizing agents generally bring about both severe damage to the hair and an oxidative destruction of the dyes formed during the coloring process from dye precursors of the developer and coupler type, so that simultaneous intense lightening and coloring effect cannot be achieved in this way. Oxidative hair coloring agents, particularly those having an additional lightening effect, are also associated with further disadvantages.

Firstly, use of oxidizing agents leads to damage to the hair structure and surface of the hair. The hair becomes brittle, its elasticity decreases, and it becomes less easy to comb. This damage increases with application time. Commercial oxidative coloring agents usually have to act on hair fibers for a period of 30 minutes and longer. Increasing the contact time leads to greater damage to the hair structure. Secondly, oxidative coloring agents generally require a basic pH range for pigment removal, particularly from pH 9.0 to pH 10.5. These pH values are required to ensure that the external cuticle opens and allows the active species (dye precursors and/or hydrogen peroxide) to penetrate into the hair. This basic environment is a further cause of damage to the hair and its structure, which likewise gains in importance as the application time increases. Splaying of the external cuticle also causes an unpleasant surface sensitivity of the hair, making it more difficult to comb when dry and wet. Furthermore, the basic pH value is frequently achieved using ammonia as the alkalizing agent, as ammonia-containing coloring agents have additional advantages in terms of coloring capacity. For the user, however, such a coloring agent not only causes additional damage to the hair, but the ammonia can also lead to irritation of the eyes or scalp, which can give rise to sensitization or even to allergic reactions. Furthermore, such coloring agents have a strong, unpleasant odor, which can also lead to irritation of the nasal mucosa. Production and storage of ammonia-containing coloring agents can also be associated with problems in terms of handling ability and stability.

The present invention therefore provides coloring agents for oxidative coloring of hair which have a significant lightening effect but which are also able to color the hair in intense and brilliant shades.

For coloring with permanent hair colors, the dye mixture must generally remain on the hair for a period of 30 to 45 minutes. For the consumer, reducing the contact time means a great simplification of the hair coloring process and increased comfort during application. Although reducing the contact time is desirable, the technical feasibility is problematic, as short application times tend to result in a less intense lightening of the hair. Accordingly, the present invention provides oxidative coloring agents having a superior lightening capacity compared to the prior art in order to reduce the contact time. Finally it is desirable to provide color-changing and/or shape-changing agents in which the irritation potential, particularly due to the addition of ammonia, is as low as possible.

Surprisingly, it has now been found that the aforementioned requirements can be satisfied to an outstanding degree by use of a combination of oxidation dye precursors as well as special bleach activators.

The invention therefore firstly provides an agent for dyeing and simultaneously lightening keratin fibers, particularly human hair, containing in a cosmetic carrier at least one oxidation dye precursor and at least one cationic 3,4-dihydroisoquinolinium derivative according to the following formula (I),

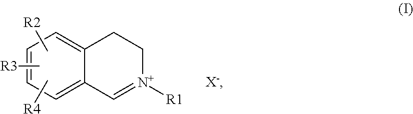

wherein
R1 is a C₁-C₆ alkyl group, a C₂-C₆ alkenyl group, a C₂-C₆ hydroxyalkyl group, a C₁-C₆ alkoxy C₂-C₆ alkyl group, a carboxy C₁-C₆ alkyl group, an aryl C₁-C₆ alkyl group, a di(C₁-C₆ alkyl)amino C₂-C₆ alkyl group, a heteroaryl C₁-C₆ alkyl group, a 3-oxobutyl group, a 2-oxopropyl group, an aryl group or a heteroaryl group,
R2, R3 and R4 are, independently of one another, a hydrogen atom, a hydroxyl group, an amino group, a di(C₁-C₆ alkyl)amino group, a C₁-C₆ alkyl group, a C₁-C₆ alkoxy group, halogen, a nitro group, a carboxy group, a nitrile group, an optionally substituted aryl group, a C₂-C₆ alkenyl group, or an optionally substituted heteroaryl group, or R2 and R3 together can form a further fused carbocyclic or heterocyclic ring which can be saturated or unsaturated and can optionally be substituted by up to three substituents, and
anion X⁻ is a physiologically tolerable anion.

The term “keratin fibers” refers to fur, wool, feathers and in particular human hair. Although the present agents are primarily suitable for dyeing keratin fibers, there is nothing in principle to preclude their use in other fields.

Agents according to the invention contain active ingredients in a cosmetic carrier. This cosmetic carrier is aqueous, alcoholic or aqueous-alcoholic. For hair dyeing, such carriers include creams, emulsions, gels or surfactant-containing foaming solutions, such as shampoos, foam aerosols or other preparations suitable for use on the hair. Aqueous-alcoholic carriers are understood to be water-containing compositions containing 3 to 70 wt. % of a C₁ to C₄ alcohol, relative to the total weight of the application mixture, particularly ethanol or isopropanol. The agents can also contain further organic solvents, such as 4-methoxybutanol, ethyl diglycol, 1,2-propylene glycol, n-propanol, n-butanol, n-butylene glycol, glycerol, diethylene glycol monoethyl ether, and diethylene glycol mono-n-butyl ether. All water-soluble organic solvents are preferred here. An aqueous carrier contains according to the invention at least 30 wt. %, particularly at least 50 wt. % of water, based on total weight of the application mixture.

As a substantial constituent, agents according to the invention contain a cationic 3,4-dihydroisoquinolinium derivative. This is a physiologically tolerable salt according to formula (I),

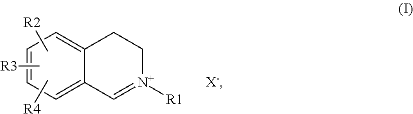

wherein
R1 is a C₁-C₆ alkyl group, a C₂-C₆ alkenyl group, a C₂-C₆ hydroxyalkyl group, a C₁-C₆ alkoxy C₂-C₆ alkyl group, a carboxy C₁-C₆ alkyl group, an aryl C₁-C₆ alkyl group, a di(C₁-C₆ alkyl)amino C₂-C₆ alkyl group, a heteroaryl C₁-C₆ alkyl group, a 3-oxobutyl group, a 2-oxopropyl group, an aryl group or a heteroaryl group,
R2, R3 and R4 are, independently of one another, a hydrogen atom, a hydroxyl group, an amino group, a di(C₁-C₆ alkyl)amino group, a C₁-C₆ alkyl group, a C₁-C₆ alkoxy group, halogen, a nitro group, a carboxy group, a nitrile group, an optionally substituted aryl group, a C₂-C₆ alkenyl group, or an optionally substituted heteroaryl group, or R2 and R3 together can form a further fused carbocyclic or heterocyclic ring, which can be saturated or unsaturated and can optionally be substituted by up to three substituents, and
anion X⁻ is a physiologically tolerable anion.

Examples of R1 and R2, R3 and R4 in formula (I) include:
for a C₁-C₆ alkyl group: methyl, ethyl, propyl, i-propyl, n-butyl, s-butyl, t-butyl, i-butyl, n-pentyl, neopentyl, particularly methyl, ethyl and i-propyl;
for a C₂-C₆ alkenyl group: ethenyl, allyl, (Z)-propen-1-yl, (E)-propen-1-yl, propen-2-yl, (E)-buten-1-yl, (Z)-buten-1-yl, (E)-but-2-en-1-yl, (Z)-but-2-en-1-yl, but-3-en-1-yl, but-2-en-2-yl, but-2-en-3-yl, pent-4-en-1-yl, hex-4-en-1-yl, particularly allyl and but-3-en-1-yl;
for a C₂-C₆ hydroxyalkyl group: CH₂CH₂OH, CH₂CH₂CH₂OH, CH₂CH(OH)CH₃, CH₂CH₂CH₂CH₂OH, particularly CH₂CH₂OH and CH₂CH₂CH₂OH;
for a C₁-C₆ alkoxy group: OCH₃, OCH₂CH₃, OCH₂CH₂CH₃, OCH(CH₃)₂, OCH₂CH₂CH₂CH₃, OCH₂CH(CH₃)₂, OCH(CH₃)CH₂CH₃, OC(CH₃)₃, particularly a methoxy or an ethoxy group;
for a C₁-C₆ alkoxy C₂-C₆ alkyl group: CH₂CH₂OCH₃, CH₂CH₂CH₂OCH₃, CH₂CH₂OCH₂CH₃, CH₂CH₂CH₂OCH₂CH₃, CH₂CH₂OCH(CH₃)₂, CH₂CH₂CH₂OCH(CH₃)₂;
for a carboxy C₁-C₆ alkyl group: CH₂CO₂H, CH₂CH₂CO₂H, CH₂CH₂CH₂CO₂H, CH₂CH(CH₃)CO₂H or one of the physiologically tolerable salts thereof;
for an aryl C₁-C₆ alkyl group: benzyl, 1-phenylethyl, 2-phenylethyl;
for a di(C₁-C₆ alkyl)amino C₂-C₆ alkyl group: CH₂CH₂N(CH₃)₂, CH₂CH₂CH₂N(CH₃)₂, CH₂CH₂CH₂CH₂N(CH₃)₂, CH₂CH(CH₃)CH₂N(CH₃)₂, CH₂CH₂CH(CH₃)N(CH₃)₂, CH₂CH₂N(CH₂CH₃)₂, CH₂CH₂CH₂N(CH₂CH₃)₂;
for a heteroaryl C₁-C₆ alkyl group: (pyridin-2-yl)methyl, (pyridin-3-yl)methyl, (pyridin-4-yl)methyl, (pyridin-2-yl)ethyl, (pyrimidin-4-yl)methyl, (imidazol-1-yl)methyl, (imidazol-2-yl)methyl, (imidazol-4-yl)methyl, (thiazol-4-yl)methyl;
for a heteroaryl group: pyridin-2-yl, pyridin-3-yl, pyridin-4-yl, pyrimidin-4-yl, imidazol-1-yl, imidazol-2-yl, imidazol-4-yl, thiazol-4-yl, thiazol-5-yl, pyrrol-1-yl, pyrrol-2-yl, pyrrol-3-yl, pyrazol-1-yl, pyrazol-3 -yl, pyrazol-4-yl, 1,2,3-triazol-1-yl, 1,2,3-triazol-4-yl; and
for an aryl group: phenyl, naphthalen-1-yl and naphthalen-2-yl.

In a particular embodiment of the present invention, R1 in formula (I) is a C₁-C₆ alkyl group, a C₂-C₆ alkenyl group or a C₂-C₆ hydroxyalkyl group.

It can further be advantageous according to the invention if R2, R3 and R4 in formula (I) are each a hydrogen atom.

Preferably, X⁻ according to formula (I) is chosen from halide (chloride, bromide, iodide), benzenesulfonate, p-toluenesulfonate, C₁-C₄ alkanesulfonate, trifluoromethanesulfonate, acetate, trifluoroacetate, perchlorate, ½ sulfate, hydrogen sulfate, tetrafluoroborate, hexafluorophosphate, hexafluorozincate or tetrafluorozincate.

It is particularly preferable for the physiologically tolerable anion X⁻ to be a halide ion (particularly chloride or bromide), hydrogen sulfate, ½ sulfate, p-toluenesulfonate, benzenesulfonate or acetate.

Particularly preferred 3,4-dihydroisoquinolinium derivatives of the general formula (I) include:

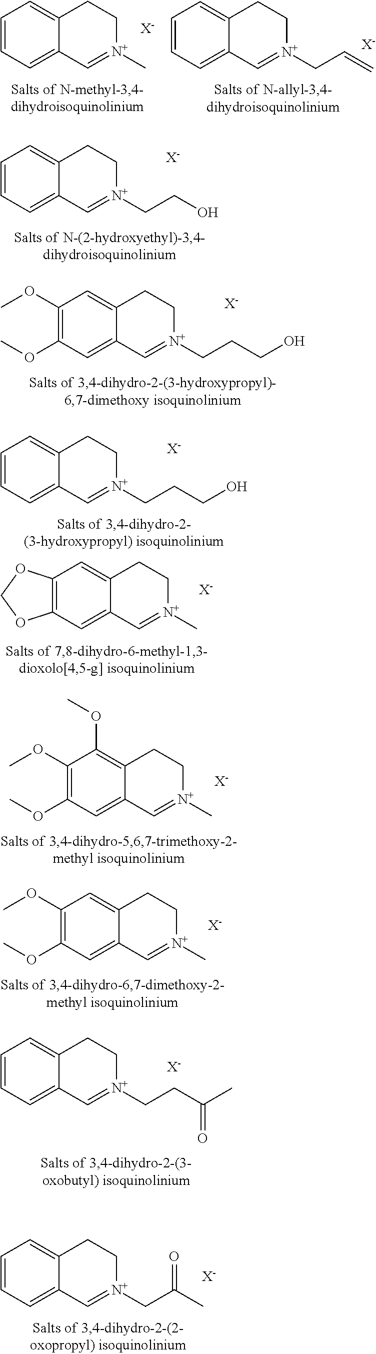

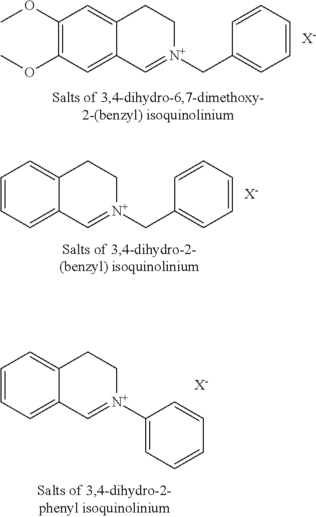

In particular, those agents are most particularly preferred which contain at least one 3,4-dihydroisoquinolinium derivative according to formula (1) chosen from N-methyl-3,4-dihydroisoquinolinium-p-toluenesulfonate, N-methyl-3,4-dihydroisoquinolinium benzenesulfonate, N-methyl-3,4-dihydroisoquinolinium bromide, N-methyl-3,4-dihydroisoquinolinium hydrogen sulfate, N-methyl-3,4-dihydroisoquinolinium acetate, N-allyl-3,4-dihydroisoquinolinium-p-toluenesulfonate, N-allyl-3,4-dihydroisoquinolinium benzenesulfonate, N-allyl-3,4-dihydroisoquinolinium bromide, N-allyl-3,4-dihydroisoquinolinium hydrogen sulfate, N-allyl-3,4-dihydroisoquinolinium acetate, 3,4-dihydro-2-(3-hydroxypropyl)isoquinolinium-p-toluenesulfonate, 3,4-dihydro-2-(3-hydroxypropyl)isoquinolinium benzenesulfonate, 3,4-dihydro-2-(3-hydroxypropyl)isoquinolinium bromide, 3,4-dihydro-2-(3-hydroxypropyl)isoquinolinium hydrogen sulfate, 3,4-dihydro-2-(3-hydroxypropyl)isoquinolinium acetate, 3,4-dihydro-2-(2-hydroxyethyl)isoquinolinium-p-toluenesulfonate, 3,4-dihydro-2-(2-hydroxyethyl)isoquinolinium benzenesulfonate, 3,4-dihydro-2-(2-hydroxyethyl)isoquinolinium bromide, 3,4-dihydro-2-(2-hydroxyethyl)isoquinolinium hydrogen sulfate, or 3,4-dihydro-2-(2-hydroxyethyl)isoquinolinium acetate.

Agents according to the invention most preferably contain N-methyl-3,4-dihydroisoquinolinium-p-toluenesulfonate as the 3,4-dihydroisoquinolinium derivative.

Agents according to the invention contain 3,4-dihydroisoquinolinium derivatives according to formula (I) in an amount of from 0.01 to 15 wt. %, preferably from 0.1 to 12 wt. % and most preferably from 0.5 to 5 wt. %, based on total weight of the ready-to-use agent.

As a further ingredient, the agents contain at least one oxidation dye precursor. Due to their mechanistic behavior during actual development of the dye, oxidation dye precursors are divided into developer precursors and coupler precursors. In a preferred embodiment the agents contain at least one oxidation dye precursor of the developer type and/or coupler type. Coloring agents preferably contain at least one developer oxidation dye precursor and at least one coupler oxidation dye precursor. The developer and coupler components are conventionally used in free form. For substances containing amino groups, however, they are preferably used in salt form, particularly in the form of hydrochlorides and hydrobromides or sulfates.

Developer components according to the invention are presented in more detail below.

p-Phenylenediamine derivatives are particularly preferred which are chosen from p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)aniline, N,N-bis-(2-hydroxyethyl)-p-phenylenediamine, 4-N,N-bis-(2-hydroxyethyl)amino-2-methylaniline, 4-N,N-bis-(2-hydroxyethyl)amino-2-chloroaniline, 2-(2-hydroxyethyl)-p-phenylenediamine, 2-(1,2-dihydroxyethyl)-p-phenylenediamine, 2-fluoro-p-phenylenediamine, 2-isopropyl-p-phenylenediamine, N-(2-hydroxypropyl)-p-phenylenediamine, 2-hydroxymethyl-p-phenylenediamine, N,N-dimethyl-3-methyl-p-phenylenediamine, N-ethyl-N-2-hydroxyethyl-p-phenylenediamine, N-(2,3-dihydroxypropyl)-p-phenylenediamine, N-(4′-aminophenyl)-p-phenylenediamine, N-phenyl-p-phenylenediamine, 2-(2-hydroxyethyloxy)-p-phenylenediamine, 2-methoxymethyl-p-phenylenediamine, 2-(2-acetylaminoethyloxy)-p-phenylenediamine, N-(2-methoxyethyl)-p-phenylenediamine, N-(4-amino-3-methylphenyl)-N-[3-(1H-imidazol-1-yl)-propyl]amine, 5,8-diaminobenzo-1,4-dioxane and the physiologically tolerable salts thereof. Most particularly preferred p-phenylenediamine derivatives according to the invention are selected from at least one compound of the group p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, N-(4-amino-3-methylphenyl)-N-[3-(1H-imidazol-1-yl)propyl]amine, 2-methoxymethyl-p-phenylenediamine and the physiologically tolerable salts of these compounds.

It can further be preferred to use as the developer component compounds containing at least two aromatic nuclei which are substituted with amino and/or hydroxyl groups. Binuclear developer components which can be used in coloring compositions according to the invention are chosen in particular from at least one of the following compounds: N,N′-bis-(2-hydroxyethyl)-N,N′-bis-(4′-aminophenyl)-1,3-diaminopropan-2-ol, N,N′-bis-(2-hydroxyethyl)-N,N′-bis-(4′-aminophenyl)ethylenediamine, N,N′-bis-(4′-aminophenyl)tetramethylenediamine, N,N′-bis-(2-hydroxyethyl)-N,N′-bis-(4′-aminophenyl)tetramethylenediamine, N,N-bis-(4-(methylamino)phenyl)tetramethylenediamine, N,N′-diethyl-N,N-bis-(4′-amino-3′-methylphenyl)ethylenediamine, bis-(2-hydroxy-5-aminophenyl)methane, N,N-bis-(4′-aminophenyl)-1,4-diazacycloheptane, N,N′-bis-(2-hydroxy-5 -aminobenzyl)piperazine, N-(4′-aminophenyl)-p-phenylenediamine and 1,10-bis-(2′,5′-diaminophenyl)-1,4,7,10-tetraoxadecane and the physiologically tolerable salts thereof. Most particularly preferred binuclear developer components are selected from N,N′-bis-(2-hydroxyethyl)-N,N′-bis-(4-aminophenyl)-1,3-diaminopropan-2-ol, bis-(2-hydroxy-5 -aminophenyl)methane, 1,3-bis-(2,5-diaminophenoxy)propan-2-ol, N,N-bis-(4-aminophenyl)-1,4-diazacycloheptane, 1,10-bis-(2,5-diaminophenyl)-1,4,7,10-tetraoxadecane or one of the physiologically tolerable salts of these compounds.

It can further be preferred to use a p-aminophenol derivative or one of its physiologically tolerable salts as the developer component. Preferred p-aminophenols include p-aminophenol, N-methyl-p-aminophenol, 4-amino-3 -methylphenol, 4-amino-3-fluorophenol, 2-hydroxymethylamino-4-aminophenol, 4-amino-3 -hydroxymethylphenol, 4-amino-2-(2-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-(2-hydroxyethyl aminomethyl)phenol, 4-amino-2-(1,2-dihydroxyethyl)phenol, 4-amino-2-fluorophenol, 4-amino-2-chlorophenol, 4-amino-2,6-dichlorophenol, 4-amino-2-(diethylaminomethyl)phenol and the physiologically tolerable salts thereof. Most particularly preferred compounds are p-aminophenol, 4-amino-3-methylphenol, 4-amino-2-aminomethylphenol, 4-amino-2-(1,2-dihydroxyethyl)phenol and 4-amino-2-(diethylaminomethyl)phenol.

The developer component can also be chosen from o-aminophenol and derivatives thereof, such as 2-amino-4-methylphenol, 2-amino-5-methylphenol or 2-amino-4-chlorophenol.

The developer component can further be chosen from heterocyclic developer components, such as pyrimidine derivatives, pyrazole derivatives, pyrazolopyrimidine derivatives and the physiologically tolerable salts thereof. Preferred pyrimidine derivatives include 2,4,5,6-tetraaminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine and 2,5,6-triaminopyrimidine. Preferred pyrazole derivatives are selected according to the invention from 4,5-diamino-1-methylpyrazole, 4,5-diamino-1-(2-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-t-butyl-1-methylpyrazole, 4,5-diamino-1-t-butyl-3-methylpyrazole, 4,5-diamino-1-(2-hydroxyethyl)-3-methylpyrazole, 4,5-diamino-1-ethyl-3-methylpyrazole, 4,5-diamino-1-ethyl-3-(4-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2-aminoethyl)amino-1,3-dimethylpyrazole, and the physiologically tolerable salts thereof, but in particular 4,5-diamino-1-(2-hydroxyethyl)pyrazole. Preferred pyrazolopyrimidine derivatives are in particular pyrazolo[1,5-a]primidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)-(2-hydroxyethyl)amino]ethanol, 2-[(7-aminopyrazolo[1,5-a]pyrimidin-3-yl)-(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 3-amino-7-dimethylamino-2,5-dimethylpyrazolo[1,5-a]pyrimidine and the physiologically tolerable salts thereof and their tautomeric forms.

Particularly preferred developer components include at least one compound from p-phenylenediamine, p-toluylenediamine, 2-(2-hydroxyethyl)-p-phenylenediamine, dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, and the physiologically tolerable salts of these compounds. Most particularly preferred developer components here are p-toluylenediamine, 2-(2-hydroxyethyl)-p-phenylenediamine, 2-methoxymethyl-p-phenylenediamine, N-(4-amino-3-methylphenyl)-N-[3-(1H-imidazol-1-yl)propyl]amine, and/or 4,5-diamino-1-(2-hydroxyethyl)pyrazole and the physiologically tolerable salts thereof The developer components are preferably used in an amount from 0.005 to 20 wt. %, preferably 0.1 to 5 wt. %, based on total ready-to-use oxidation coloring agent.

With respect to oxidative dyeing, coupler components develop no significant color on their own but always need the presence of developer components. It is therefore preferable that, with the use of at least one coupler component, at least one developer component is additionally used.

Coupler components within the meaning of the invention permit at least one substitution of a chemical residue of the coupler with the oxidized form of the developer component. A covalent bond forms between the coupler and developer component in this process.

Preferred m-aminophenol coupler components include 3-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-diethylaminophenol, N-cyclopentyl-3-aminophenol, 1,3-dihydroxy-5-(methylamino)benzene, 3-ethylamino-4-methylphenol, 2,4-dichloro-3-aminophenol and the physiologically tolerable salts of all aforementioned compounds.

The m-diaminobenzenes or derivatives thereof for use according to the invention include m-phenylenediamine, 2-(2,4-diaminophenoxy)ethanol, 1,3-bis(2,4-diaminophenoxy)propane, 1-methoxy-2-amino-4-(2′-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2′-hydroxyethyl)aminobenzene and the physiologically tolerable salts of all aforementioned compounds.

The o-diaminobenzenes or derivatives thereof for use according to the invention include 3,4-diaminobenzoic acid and 2,3-diamino-1-methylbenzene and the physiologically tolerable salts of all aforementioned compounds.

Preferred di- or trihydroxybenzenes and derivatives thereof include resorcinol, resorcinol monomethyl ether, 2-methylresorcinol, 5-methylresorcinol, 2,5-dimethylresorcinol, 2-chlororesorcinol, 4-chlororesorcinol, pyrogallol and 1,2,4-trihydroxybenzene.

Pyridine derivatives for use according to the invention include 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 3,4-diaminopyridine, 2-(2-methoxyethyl)amino-3 -amino-6-methoxypyridine, 2-(4′-methoxyphenyl)amino-3-aminopyridine, and the physiologically tolerable salts of the aforementioned compounds.

Preferred naphthalene derivatives containing at least one hydroxyl group include 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1,6-dihydroxynaphthalene, 1,7-dihydroxynaphthalene, 1,8-dihydroxynaphthalene, 2,7-dihydroxynaphthalene and 2,3-dihydroxynaphthalene.

Indole derivatives for use according to the invention include 4-hydroxyindole, 6-hydroxyindole and 7-hydroxyindole and the physiologically tolerable salts of the aforementioned compounds. The indoline derivatives for use according to the invention include 4-hydroxyindoline, 6-hydroxyindoline and 7-hydroxyindoline and the physiologically tolerable salts of the aforementioned compounds.

Preferred pyrimidine derivatives include 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine and the physiologically tolerable salts of the aforementioned compounds.

Particularly preferred coupler components according to the invention include 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethypamino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds or the physiologically tolerable salts of the aforementioned compounds. Resorcinol, 2-methylresorcinol, 5-amino-2-methylphenol, 3-aminophenol, 2-(2,4-diaminophenoxy)ethanol, 1,3-bis(2,4-diaminophenoxy)propane, 1-methoxy-2-amino-4-(2′-hydroxyethylamino)benzene, 2-amino-3-hydroxypyridine and 1-naphthol and one of the physiologically tolerable salts thereof are most particularly preferred.

Coupler components are preferably used in an amount from 0.005 to 20 wt. %, preferably 0.1 to 5 wt. %, based on total weight of the ready-to-use oxidation coloring agent.

In the context of the present invention the following combinations of oxidation dye precursors of the developer type and the coupler type are particularly preferred. Further dye precursors can however also be combined with the oxidation dye precursors cited as combinations: p-toluylenediamine/resorcinol; p-toluylenediamine/2-methylresorcinol; p-toluylenediamine/5-amino-2-methylphenol; p-toluylenediamine/3-aminophenol; p-toluylenediamine/2-(2,4-diaminophenoxy)ethanol; p-toluylenediamine/1,3-bis-(2,4-diaminophenoxy)propane; p-toluylenediamine 1-methoxy-2-amino-4-(2-hydroxyethylamino)benzene; p-toluylenediamine/2-amino-3-hydroxypyridine; p-toluylenediamine/1-naphthol; 2-(2-hydroxyethyl)-p-phenylenediamine/resorcinol; 2-(2-hydroxyethyl)-p-phenylenediamine 2-methylresorcinol; 2-(2-hydroxyethyl)-p-phenylenediamine/5-amino-2-methylphenol; 2-(2-hydroxyethyl)-p-phenylenediamine/3-aminophenol; 2-(2-hydroxyethyl)-p-phenylenediamine/2-(2,4-diaminophenoxy)ethanol; 2-(2-hydroxyethyl)-p-phenylenediamine/1,3-bis-(2,4-diaminophenoxy)propane; 2-(2-hydroxyethyl)-p-phenylenediamine/1-methoxy-2-amino-4-(2-hydroxyethylamino)benzene; 2-(2-hydroxyethyl)-p-phenylenediamine/2-amino-3-hydroxypyridine; 2-(2-hydroxyethyl)-p-phenylenediamine/1-naphthol; 2-methoxymethyl-p-phenylenediamine/resorcinol; 2-methoxymethyl-p-phenylenediamine/2-methylresorcinol; 2-methoxymethyl-p-phenylenediamine/5-amino-2-methylphenol; 2-methoxymethyl-p-phenylenediamine/3-aminophenol; 2-methoxymethyl-p-phenylenediamine/2-(2,4-diaminophenoxy)ethanol; 2-methoxymethyl-p-phenylenediamine/1,3-bis(2,4-diaminophenoxy)propane; 2-methoxymethyl-p-phenylenediamine/1-methoxy-2-amino-4-(2-hydroxyethylamino)benzene; 2-methoxymethyl-p-phenylenediamine/2-amino-3-hydroxypyridine; 2-methoxymethyl-p-phenylenediamine/1-naphthol; N-(4-amino-3-methylphenyl)-N-[3-(1H-imidazol-1-yl)propyl]amine/resorcinol; N-(4-amino-3-methylphenyl)-N-[3-(1H-imidazol-1-yl)propyl]amine/2-methylresorcinol; N-(4-amino-3-methylphenyl)-N-[3-(1H-imidazol-1-yl)propyl]amine/5-amino-2-methylphenol; N-(4-amino-3-methylphenyl)-N-[3-(1H-imidazol-1-yl)propyl]amine/3-aminophenol; N-(4-amino-3-methylphenyl)-N-[3-(1H-imidazol-1-yl)propyl]amine/2-(2,4-diaminophenoxy)ethanol; N-(4-amino-3-methylphenyl)-N-[3-(1H-imidazol-1-yl)propyl]amine/1,3-bis(2,4-diaminophenoxy)propane; N-(4-amino-3-methylphenyl)-N-[3-(1H-imidazol-1-yl)propyl]amine/1-methoxy-2-amino-4-(2-hydroxyethylamino)benzene; N-(4-amino-3-methylphenyl)-N-[3-(1H-imidazol-1-yl)propyl]amine/2-amino-3-hydroxypyridine; N-(4-amino-3-methylphenyl)-N-[3-(1H-imidazol-1-yl)propyl]amine/1-naphthol; 4,5-diamino-1-(2-hydroxyethyl)pyrazole/resorcinol; 4,5-diamino-1-(2-hydroxyethyl)pyrazole/2-methylresorcinol; 4,5-diamino-1-(2-hydroxyethyl)pyrazole/5-amino-2-methylphenol; 4,5-diamino-1-(2-hydroxyethyl)pyrazole/3-aminophenol; 4,5-diamino-1-(2-hydroxyethyl)pyrazole/2-(2,4-diaminophenoxy)ethanol; 4,5-diamino-1-(2-hydroxyethyl)pyrazole/1,3-bis(2,4-diaminophenoxy)propane; 4,5-diamino-1-(2-hydroxyethyl)pyrazole/1-methoxy-2-amino-4-(2-hydroxyethylamino)benzene; 4,5-diamino-1-(2-hydroxyethyl)pyrazole 2-amino-3-hydroxypyridine; 4,5-diamino-1-(2-hydroxyethyl)pyrazole/1-naphthol.

It is most particularly advantageous if the agents contain at least one of the above oxidation dye precursor combinations together with N-methyl-3,4-dihydroisoquinolinium-p-toluenesulfonate as the 3,4-dihydroisoquinolinium derivative.

According to the invention, oxidation dye precursors are preferably used in an amount from 0.005 to 20 wt. %, preferably 0.05 to 5 wt. % and particularly preferably 0.1 to 5 wt. %, based on total weight of the ready-to-use oxidation coloring agent. Developer components and coupler components are generally used in approximately molar amounts to one another. Even if molar use has proved convenient, a certain excess of individual oxidation dye precursors is not disadvantageous, so that developer components and coupler components can be in a molar ratio of 1 to 0.5 to 1 to 3, in particular 1 to 1 to 1 to 2.

In a further embodiment the agent can also contain at least one further coloring component in addition to the oxidation dye precursors.

This further coloring component is preferably chosen from oxo dye precursors and/or from at least one precursor of nature-analogous dyes and/or from at least one direct dye.

In a further embodiment oxo dye precursors can also be used as the color-changing component. Oxo dye precursors are preferably used as a combination of at least one compound containing at least one reactive carbonyl group and at least one compound chosen from CH-acid compounds and/or from compounds containing a primary or secondary amino group or hydroxyl group, chosen from primary or secondary aromatic amines, nitrogen-containing heterocyclic compounds and aromatic hydroxyl compounds. If they are used, the aforementioned compounds are preferably used in an amount from 0.01 to 5 wt. %, in particular 0.1 to 3 wt. %, relative to the total weight of the complete agent.

In a further embodiment, the color-changing component is chosen from dye precursors of nature-analogous dyes. Indoles and indolines containing at least two groups selected from hydroxyl and/or amino groups, preferably as a substituent on the six-membered ring, are preferably used as dye precursors of nature-analogous dyes. Dye precursors of nature-analogous dyes are preferably used in an amount from 0.001 to 5 wt. %, relative to the entire application preparation. The total amount of direct dyes is preferably at most 3 wt. %. Particularly suitable as precursors of nature-analogous hair dyes are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline and 5,6-dihydroxyindoline-2-carboxylic acid, in particular 5,6-dihydroxyindoline. Outstandingly suitable as precursors of nature-analogous hair dyes are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, in particular 5,6-dihydroxyindole.

Agents according to the invention can also contain at least one direct dye as the color-changing component. These are dyes which attach directly to the hair and require no oxidative process to develop the color. Direct dyes are conventionally nitrophenylene diamines, nitroaminophenols, azo dyes, anthraquinones or indophenols. Direct dyes are preferably used in an amount from 0.001 to 20 wt. %, relative to the entire application preparation. The total amount of direct dyes is preferably at most 20 wt. %. Direct dyes can be divided into anionic, cationic and non-ionic direct dyes.

Preferred anionic direct dyes are compounds known under the international names or trade names Tetrabromophenol Blue, Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1 and Acid Black 52.

Preferred cationic direct dyes are cationic triphenylmethane dyes, such as Basic Blue 7, Basic Blue 26, Basic Violet 2 and Basic Violet 14, aromatic systems which are substituted with a quaternary nitrogen group, such as Basic Yellow 57, Basic Red 76, Basic Blue 99, Basic Brown 16 and Basic Brown 17, as well as direct dyes containing a heterocyclic compound having at least one quaternary nitrogen atom. Compounds also known under the names Basic Yellow 87, Basic Orange 31 and Basic Red 51 are most particularly preferred cationic direct dyes.

Non-ionic nitro and quinone dyes and neutral azo dyes in particular are suitable as non-ionic direct dyes. Preferred non-ionic direct dyes are the compounds known under the international names or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-4-nitrophenol. Naturally occurring dyes, such as are contained for example in henna red, henna neutral, henna black, chamomile flowers, sandalwood, black tea, alder buckthorn bark, sage, logwood, madder root, catechu and alkanet root, can also be used as direct dyes.

It is not necessary for oxidation dye precursors, oxo dye precursors, direct dyes or nature-analogous dyes to be uniform compounds. Instead, the individual dyes can also contain small amounts of further components arising from the manufacturing processes for the individual dyes, provided that they do not adversely influence the dyeing result or need to be excluded for other reasons (e.g., toxicological).

Packaging of the coloring agents according to the invention is not generally subject to any restrictions. The agents are conventionally packaged as one-component agents (A), which are optionally mixed immediately before application with a second preparation containing an oxidizing agent, for example. In some cases, however, it has also proved preferable for the product to be packaged as a two-component agent. In a preferred embodiment, the agents are packaged so that the 3,4-dihydroisoquinolinium derivative as one of the components substantial to the invention is packaged in a preparation (A2) separate from preparation (A1) containing the oxidation dye precursor. These two preparations are mixed together before application, and an oxidizing agent preparation (B) can optionally be added. It can be advantageous firstly to mix the oxidizing agent preparation (B) and preparation (A2) containing the 3,4-dihydroisoquinolinium derivative, and then add preparation (A1) containing the oxidation dye precursor. It is likewise possible firstly to mix preparation (A2) containing the 3,4-dihydroisoquinolinium derivative and preparation (A1) containing the oxidation dye precursor, and then add the oxidizing agent preparation (B).

For oxidative colorings, the color can develop with atmospheric oxygen. A chemical oxidizing agent is preferably used, however, particularly if a lightening effect on human hair is sought in addition to the coloring effect. This lightening effect may be sought regardless of the dyeing method. Suitable oxidizing agents include persulfates, peroxodisulfates, chlorites, hypochlorites, and, in particular, hydrogen peroxide and/or one of its solid addition products with organic or inorganic compounds.

In order to avoid a premature, undesired reaction of the oxidation dye precursors due to the oxidizing agent, oxidation dye precursors and oxidizing agents are conveniently packaged separately from one another and brought into contact only immediately before application.

The present invention therefore also provides an agent which is prepared directly before application by mixing at least two preparations, the at least two preparations being provided in at least two separately packaged containers, with one container containing a coloring agent (A) which has in a cosmetic carrier at least one oxidation dye precursor and at least one 3,4-dihydroisoquinolinium derivative according to formula (I), and the second container containing an oxidizing agent preparation (B) having at least one oxidizing agent.

A particular embodiment of the present invention is an agent which is prepared directly before application by mixing at least three preparations, with the at least three preparations being provided in at least three separately packaged containers and one container containing a coloring agent (A1) having in a cosmetic carrier at least one oxidation dye precursor, a second container containing an agent (A2) having in a cosmetic carrier at least one 3,4-dihydroisoquinolinium derivative according to formula (I), and a third container containing an oxidizing agent preparation (B) having at least one oxidizing agent.

Oxidizing agent preparation (B) preferably contains as the oxidizing agent hydrogen peroxide and/or one of its solid addition products with organic or inorganic compounds, such as urea, melamine and sodium borate. The amount of hydrogen peroxide in the ready-to-use agent is preferably 0.5 to 12 wt. %, preferably 2 to 10 wt. %, particularly preferably 3 to 6 wt. % (calculated as H₂O₂ 100%), based on total weight of the ready-to-use agent.

Such oxidizing agent preparations are preferably aqueous, free-flowing oxidizing agent preparations. Preferred preparations are those wherein the free-flowing oxidizing agent preparation contains—relative to its weight—40 to 90 wt. %, preferably 50 to 85 wt. %, more preferably 55 to 80 wt. %, even more preferably 60 to 77.5 wt. %, and particularly 65 to 75 wt. % of water.

According to the invention, the oxidization coloring agent can also be applied to the hair together with a catalyst which activates oxidation of the dye precursors, for example, through atmospheric oxygen. Such catalysts include certain enzymes, iodides, quinones or metal ions. Suitable enzymes are, for example, peroxidases, which can significantly strengthen the action of small amounts of hydrogen peroxide. Use of certain metal ions or complexes can likewise be preferred in order to obtain intense, long-lasting colors. Suitable metal ions include Zn²⁺, Cu²⁺, Fe²⁺, Fe³⁺, Mn²⁺, Mn⁴⁺, Li⁺, Mg²⁺, Ca²⁺, Ce⁴⁺, V³⁺, Co²⁺, Ru³⁺ and AI³⁺.

Preferably, the oxidizing agent preparations (b) contain at least one stabilizer or complexing agent. Particularly preferred stabilizers are phenacetin, alkali benzoates (sodium benzoate) and salicylic acid. Also preferred is the use of complexing agents. Complexing agents are substances which are capable of complexing metal ions. Preferred complexing agents are chelate complexing agents. Preferred complexing agents according to the invention are nitrogen-containing polycarboxylic acids, particularly EDTA, and phosphonates, preferably hydroxyalkane or aminoalkane phosphonates and in particular 1-hydroxyethane-1,1-diphosphonate (HEDP) and/or ethylenediamine tetramethylene phosphonate (EDTMP) and/or diethylenetriamine pentamethylene phosphonate (DTPMP) or the sodium salts thereof.

In addition to the actual oxidizing agent, the oxidizing agent preparation contains further auxiliary substances and additives. It is thus preferable for the oxidizing agent preparation to contain at least one thickening agent. There are no general restrictions regarding these thickening agents. Both organic and purely inorganic thickening agents can be used.

To further increase the lightening capacity, at least one optionally hydrated SiO₂ compound can optionally be added to the composition according to the invention. Although even small amounts of hydrated SiO₂ compounds increase the lightening capacity, it can be preferable to use hydrated SiO₂ compounds in amounts from 0.05 wt. % to 15 wt. %, preferably in amounts from 0.15 wt. % to 10 wt. % and most preferably in amounts from 0.2 wt. % to 5 wt. %, relative to the anhydrous composition according to the invention. The specified amounts indicate the content of SiO₂ compounds (excluding their water component) in the agents. The present invention is generally not subject to any restrictions regarding the optionally hydrated SiO₂ compounds. Silicic acids, oligomers and polymers thereof, as well as salts thereof are preferred. Preferred salts are the alkali salts, particularly the potassium and sodium salts. The sodium salts are most particularly preferred. The optionally hydrated SiO₂ compounds can be present in various forms. According to the invention, the SiO₂ compounds are preferably used in the form of silica gels or particularly preferably as water glass. In some cases these SiO₂ compounds can be present in aqueous solution. Most particularly preferred are water glasses formed from a silicate of the formula (SiO₂)ₙ(Na₂O)ₘ(K₂O)ₚ, where n is a positive rational number and m and p are, independently of each other, a positive rational number or 0, with the provisos that at least one of the parameters m or p is different from 0 and that the ratio of n to the sum of m and p is from 1:4 to 4:1. Particularly preferred water glasses are sold inter alia by Henkel under the names Ferrosil® 119, soda water glass 40/42, Portil® A, Portil® AW and Portil® W and by Akzo Nobel under the name Britesil® C20.

Oxidizing agent preparation (B) and/or preparation (A) or preparations (A1) and (A2) are preferably packaged as free-flowing preparations. An emulsifier or surfactant is preferably also added to the free-flowing preparations (A) or (A1) and (A2) and/or (B), surface-active substances being referred to as surfactants or as emulsifiers depending on the area of application and being selected from anionic, cationic, zwitterionic, amphoteric and non-ionic surfactants and emulsifiers.

All anionic surface-active substances suitable for use on the human body are suitable as anionic surfactants in preparations according to the invention. Preferred anionic surfactants are alkyl sulfates, alkyl ether sulfates and ether carboxylic acids with 10 to 18 C atoms in the alkyl group and up to 12 glycol ether groups in the molecule.

Surface-active compounds classed as zwitterionic surfactants are those bearing at least one quaternary ammonium group and at least one carboxylate, sulfonate or sulfate group in the molecule. Particularly suitable zwitterionic surfactants are betaines such as N-alkyl-N,N-dimethylammonium glycinates, N-acyl aminopropyl-N,N-dimethylammonium glycinates each having 8 to 18 C atoms in the alkyl or acyl group and cocacyl aminoethyl hydroxyethyl carboxymethyl glycinate. A preferred zwitterionic surfactant is the fatty acid amide derivative known under the INCI name Cocamidopropyl Betaine.

Examples of suitable amphoteric surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred amphoteric surfactants are N-cocalkyl aminopropionate, cocacyl aminoethyl aminopropionate and C₁₂-C₁₈ acyl sarcosine.

It has also proved advantageous for coloring and lightening agents according to the invention to also contain non-ionogenic interfacially-active substances. In particular, C₈ to C₂₂ alkyl mono- and oligoglycosides and ethoxylated analogs thereof are suitable as non-ionic surfactants. In particular, non-ethoxylated compounds have proved to be particularly suitable. Alkylene oxide addition products with saturated linear fatty alcohols and fatty acids each containing 2 to 30 mol of ethylene oxide per mol of fatty alcohol or fatty acid have proved to be further preferred non-ionic surfactants. Preparations having outstanding properties are likewise obtained if they contain fatty acid esters of ethoxylated glycerol as non-ionic surfactants.

Anionic, non-ionic, zwitterionic or amphoteric surfactants are used in amounts from 0.1 to 45 wt. %, preferably 1 to 30 wt. % and most particularly preferably 1 to 15 wt. %, relative to the total amount of the ready-to-use agent.

Likewise preferred according to the invention are cationic surfactants of the quaternary ammonium compound, esterquat and amidoamine type. Preferred quaternary ammonium compounds are ammonium halides, in particular chlorides and bromides, such as alkyltrimethylammonium chlorides, dialkyldimethylammonium chlorides and trialkylmethylammonium chlorides, as well as imidazolium compounds known under the INCI names Quatemium-27 and Quaternium-83. Other cationic surfactants which can be used are quaternized protein hydrolysates. The alkylamidoamines are conventionally produced by amidation of natural or synthetic fatty acids and fatty acid cuts with dialkyl amino amines. A compound from this group of substances that is particularly suitable is the stearamidopropyl dimethylamine commercially available under the name Tegoamid® S 18. Likewise, very readily biodegradable are quaternary ester compounds known as esterquats. Preferred esterquats are quaternized ester salts of fatty acids with triethanolamine, quaternized ester salts of fatty acids with diethanol alkyl amines and quaternized ester salts of fatty acids with 1,2-dihydroxypropyl dialkylamines. Such products are sold under the trademarks Stepantex®, Dehyquart® and Armocare®, for example. Cationic surfactants are used in the agents preferably in amounts of 0.05 to 10 wt. %, relative to the total agent. Amounts of 0.1 to 5 wt. % are particularly preferred.

In a preferred embodiment non-ionic, zwitterionic and/or amphoteric surfactants and mixtures thereof are preferred.

In a further preferred embodiment, the action of the active ingredient is increased by addition of emulsifiers. Such emulsifiers include addition products of 4 to 30 mol of ethylene oxide and/or 0 to 5 mol of propylene oxide with linear fatty alcohols having 8 to 22 C atoms, with fatty acids having 12 to 22 C atoms and with alkyl phenols having 8 to 15 C atoms in the alkyl group; C₁₂-C₂₂ fatty acid monoesters and diesters of addition products of 1 to 30 mol of ethylene oxide with polyols having 3 to 6 carbon atoms, in particular with glycerol; C₈-C₂₂ alkyl mono- and oligoglycosides and ethoxylated analogs thereof, with degrees of oligomerization of 1.1 to 5, in particular 1.2 to 2.0, and glucose as the sugar component being preferred; addition products of 5 to 60 mol of ethylene oxide with castor oil and hydrogenated castor oil; sterols (zoosterols, phytosterols, mycosterols); phospholipids; fatty acid esters of sugars and sugar alcohols such as sorbitol; polyglycerols and polyglycerol derivatives as well as linear and branched fatty acids having 8 to 30 C atoms and Na, K, ammonium, Ca, Mg and Zn salts thereof.

Agents according to the invention preferably contain emulsifiers in amounts from 0.1 to 25 wt. %, particularly 0.5 to 15 wt. %, relative to the total amount of the ready-to-use agent. The compositions can preferably contain at least one non-ionogenic emulsifier or surfactant having an HLB value of 8 to 18 according to the definition given in Rompp-Lexikon Chemie (Ed. J. Falbe, M. Regitz), 10^{th} Edition, Georg Thieme Verlag Stuttgart, New York (1997), p. 1764. Non-ionogenic emulsifiers or surfactants having an HLB value of 10 to 15 can be particularly preferred according to the invention.

Agents according to the invention can additionally contain further active ingredients, auxiliary substances and additives, such as non-ionic polymers; silicones such as volatile or non-volatile, straight-chain, branched or cyclic, crosslinked or uncrosslinked polyalkylsiloxanes (such as dimethicone or cyclomethicone), polyarylsiloxanes and/or polyalkylarylsiloxanes; cationic polymers; zwitterionic and amphoteric polymers; anionic polymers such as polyacrylic acids; other thickening agents such as agar-agar, guar gum, alginates, xanthan gum, gum arabic, karaya gum, carob seed meal, linseed gums, dextrans, cellulose derivatives, for example methyl cellulose, hydroxyalkyl cellulose and carboxymethyl cellulose, starch fractions and derivatives such as amylose, amylopectin and dextrins, clays such as bentonite or fully synthetic hydrocolloids such as polyvinyl alcohol; texturizing agents such as glucose, maleic acid and lactic acid; hair-conditioning compounds such as phospholipids, for example soya lecithin, egg lecithin and cephalins as well as silicone oils; protein hydrolysates, in particular elastin, collagen, keratin, milk protein, soya protein and wheat protein hydrolysates, condensation products thereof with fatty acids as well as quatemized protein hydrolysates; perfume oils, dimethyl isosorbide and cyclodextrin; active ingredients to improve the fiber structure, in particular mono-, di- and oligosaccharides such as glucose, galactose, fructose, fruit sugars and lactose; defoaming agents such as silicones; dyes to color the agent; anti-dandruff active ingredients; amino acids and oligopeptides, in particular arginine and/or serine; light stabilizers; active ingredients such as panthenol, pantothenic acid, pantolactone, allantoin, pyrrolidinone carboxylic acids and salts thereof as well as bisabolol; polyphenols, in particular hydroxycinnamic acids, 6,7-dihydroxycoumarin, hydroxybenzoic acids, catechins, tannins, leukoanthocyanidins, anthocyanidins, flavanones, flavones and flavonols; ceramides, preferably sphingolipids or pseudoceramides; vitamins, provitamins and vitamin precursors, in particular those of groups A, B₃, B₅, B₆, C, E, F and H; plant extracts such as extracts from green tea, oak bark, stinging nettle, witch hazel, hops, chamomile, burdock, horsetail, whitethom, lime blossom, almond, aloe vera, pine, horse chestnut, sandalwood, juniper, coconut, mango, apricot, lemon, lychee, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root; cholesterol; consistency modifiers such as sugar esters, polyol esters or polyol alkyl ethers; fats and waxes such as fatty alcohols, beeswax, montan wax and paraffins; swelling and penetrating substances such as glycerol, propylene glycol monoethyl ethers, carbonates, hydrogen carbonates, guanidines, ureas as well as primary, secondary and tertiary phosphates; opacifiers such as latex, styrene/PVP and styrene/acrylamide copolymers; pearlescent agents; pigments; blowing agents such as propane-butane mixtures, N₂O, dimethyl ether, CO₂ and air, as well as antioxidants.

One skilled in the art will select these further substances according to the desired properties of the agents. Regarding further optional components and the amounts of these components used, reference is expressly made to relevant manuals known to the person skilled in the art, for example, Kh. Schrader, Grundlagen and Rezepturen der Kosmetika, 2^{nd} Edition, Hüthig Buch Verlag, Heidelberg, 1989. The additional active ingredients and auxiliary substances are used in the agents preferably in amounts of 0.0001 to 10 wt. % in each case, in particular 0.0005 to 5 wt. %, relative to the total weight of the application mixture.

If a strong lightening effect is desired, preferably a lightening preparation (C) containing at least one further bleaching strength intensifier is incorporated in the mixture comprising the oxidizing agent preparation (B) and preparation (A) containing at least one oxidation dye precursor and at least one 3,4-dihydroisoquinolinium derivative according to formula (I).

It makes no difference whether a mixture of (A) and (B) is prepared first and then the lightening preparation (C) is added, or whether a different sequence of mixing the individual components is used. It is preferable to mix the individual preparations in as short a time sequence as possible and to apply the agent preferably contemporaneously to the keratin fibers.

A further embodiment of the present application is therefore an agent for bleaching and dyeing keratin fibers wherein it is prepared prior to application by mixing at least one oxidizing agent preparation (B) containing at least one oxidizing agent chosen from hydrogen peroxide or its addition products on solid carriers, at least one lightening preparation (C) containing at least one bleaching strength intensifier, and at least one preparation (A) containing in a cosmetic carrier at least one oxidation dye precursor and at least one cationic 3,4-dihydroisoquinolinium derivative according to formula (I).

As mentioned above it can be advantageous to use, instead of preparation (A) containing in a cosmetic carrier at least one oxidation dye precursor and at least one cationic 3,4-dihydroisoquinolinium derivative according to formula (I), two separate preparations (A1) containing in a cosmetic carrier at least one oxidation dye precursor and (A2) containing in a cosmetic carrier at least one cationic 3,4-dihydroisoquinolinium derivative according to formula (I).

A further embodiment of the present application is therefore an agent for bleaching and dyeing keratin fibers wherein it is prepared prior to application by mixing at least one oxidizing agent preparation (B) containing at least one oxidizing agent selected from hydrogen peroxide or its addition products on solid carriers, at least one lightening preparation (C), containing at least one bleaching strength intensifier, and at least one preparation (A1) containing in a cosmetic carrier at least one oxidation dye precursor and at least one preparation (A2) containing in a cosmetic carrier at least one cationic 3,4-dihydroisoquinolinium derivative according to formula (I). The sequence in which preparations (A1), (A2), (B) and (C) are mixed is irrelevant. It is however advantageous according to the invention to mix preparations (A1) and (A2) first and then to add the further preparations, preferably as a premix consisting of (B) and (C).

For strong lightening of very dark hair, use of hydrogen peroxide or its addition products with organic or inorganic compounds alone is often not sufficient. Agents according to the invention can therefore additionally contain further lightening and/or bleaching agents.

Should the consumer desire a very strong lightening effect, it can therefore be preferable in a further embodiment if the coloring agent additionally contains as the lightening preparation (C) at least one inorganic peroxo compound for lightening the keratin fibers. The inorganic peroxo compound is preferably chosen from ammonium persulfate, alkali metal persulfates, ammonium peroxomonosulfate, alkali metal hydrogen peroxomonosulfates, alkali metal peroxodiphosphates and alkaline-earth metal peroxides. Particularly preferred inorganic peroxo compounds as bleaching strength intensifiers are ammonium peroxodisulfate, potassium peroxodisulfate, sodium peroxodisulfate, potassium hydrogen peroxomonosulfate, potassium peroxodiphosphate, magnesium peroxide and barium peroxide. Preferred inorganic peroxo compounds are peroxodisulfate salts, in particular ammonium peroxodisulfate, potassium peroxodisulfate and sodium peroxodisulfate. The peroxodisulfate salts can be included in an amount from 0.1 to 25 wt. %, in particular in an amount from 0.5 to 15 wt. %, relative to the total weight of the ready-to-use agent. Use of persulfate salts or peroxodisulfate salts generally takes place in the form of an optionally dedusted powder or a pressed molding.

It can, however, be advantageous if the agents are free from inorganic peroxo compounds. The agents can contain a further bleaching strength intensifier instead of and/or in addition to the solid peroxo compounds. Compounds which under perhydrolysis conditions give rise to aliphatic peroxocarboxylic acids and/or substituted perbenzoic acid, carbonic derivatives, alkyl carbonates and carbomates as well as silyl carbonates and carbamates can be used as additional bleaching strength intensifiers. Useful bleaching strength intensifiers include nitrogen-containing heterocyclic compounds. Imidazole can be cited in particular as an example of a nitrogen-containing heterocyclic bleaching strength intensifier. Bleaching strength intensifiers used in addition to or instead of peroxo compounds are included in the cosmetic agents preferably in amounts from 0.05 to 10 wt. %, in particular in amounts from 0.2 to 5 wt. %, relative in each case to the total weight of the ready-to-use agent.

While there may be no restrictions regarding formulation of the lightening preparation (C), preferably preparation (C) is formulated free of water. Free of water means a water content relative to preparation (C) of less than 5 wt. %, in particular less than 2 wt. %. Lightening preparations containing less than 0.1 wt. % of water can be most particularly preferred according to the invention. Preparation (C) is preferably formulated as a powder or as an anhydrous paste. Where it is formulated as an anhydrous paste it has proved particularly preferable for the preparation (C) to contain at least one non-hydroxylated fatty acid ester with a melting point of at most 50° C., in particular at most 30° C., and/or at least one C₁₀-C₃₀ fatty acid with at least one additional hydroxyl group and/or a derivative thereof. Esters of non-hydroxylated C₆ to C₃₀ alkyl monocarboxylic acids with C₂ to C₃₀ monoalcohols are preferably suitable according to the invention as fatty acid esters. The monoesters of fatty acids with monoalcohols having 2 to 24 C atoms are preferred.

A preferred embodiment of the present invention is one where the ready-to-use agent has a pH of from 7 to 11, particularly from 8 to 10.5, more particularly from 8.5 to 10.0. The pH values are pH values measured at a temperature of 22° C. The pH is conventionally adjusted with pH adjusters. Acidifying and alkalizing agents commonly used in cosmetics for adjusting the pH are familiar to the person skilled in the art. Alkalizing agents which can be used for adjusting the pH are typically chosen from inorganic salts, in particular alkali and alkaline-earth metals, organic alkalizing agents, in particular amines, basic amino acids and alkanol amines, and ammonia. Preferred acidifying agents according to the invention are food acids, such as citric acid, acetic acid, malic acid or tartaric acid, as well as dilute mineral acids. Particularly preferred alkanol amines are monoethanolamine and triethanolamine. The alkalizing agents are preferably included in an amount from 0.05 to 10 wt. %, in particular 0.5 to 5 wt. %, relative in each case to the total weight of the ready-to-use agent.

It has been demonstrated that the lightening effect of the 3,4-dihydroisoquinolinium derivatives is positively influenced by addition of triethanolamine. A particularly preferred embodiment of the present application is therefore one where the agent contains triethanolamine as a further additional component. Triethanolamine is preferably added in an amount from 0.05 to 10 wt. %, in particular 0.5 to 5 wt. %, relative in each case to the total weight of the ready-to-use agent.

As has already been mentioned, the agents can also be prepared directly before application from two or more separately packaged preparations. This allows separation of incompatible constituents to prevent a premature reaction. Separation into multicomponent systems is particularly appropriate in cases where there is a risk or a likelihood of incompatibilities between the constituents. In such systems, the ready-to-use agent is prepared by the consumer by mixing the components directly before application. A coloring and/or lightening agent in which the oxidation dye precursors are initially separate from the oxidizing agent preparation preferably containing hydrogen peroxide is preferred here.

A preferred packaging format of the agent according to the invention is a kit of parts containing in separately packaged containers
at least one oxidizing agent preparation (B) containing at least one oxidizing agent, and
at least one preparation (A), wherein preparation (A) contains in a cosmetic carrier at least one oxidation dye precursor and at least one 3,4-dihydroisoquinolinium derivative according to formula (I).

A further preferred packaging format of the agent is a kit of parts which contains in separately packaged containers
at least one oxidizing agent preparation (B) containing at least one oxidizing agent,
at least one preparation (Al) which contains in a cosmetic carrier at least one oxidation dye precursor, and
at least one preparation (A2), wherein preparation (A2) contains in a cosmetic carrier at least one 3,4-dihydroisoquinolinium derivative according to formula (I).

If a particularly strong lightening effect is desired, a preferred further packaging format of the agent is a kit of parts containing in separately packaged containers
at least one oxidizing agent preparation (B) containing at least one oxidizing agent,
at least one lightening preparation (C) containing at least one bleaching strength intensifier, and
at least one preparation (A), wherein preparation (A) contains in a cosmetic carrier at least one oxidation dye precursor and at least one 3,4-dihydroisoquinolinium derivative according to formula (I).

Finally, a further preferred packaging format of the agent for a particularly strong lightening effect is a kit of parts containing in separately packaged containers
at least one oxidizing agent preparation (B) containing at least one oxidizing agent,
at least one lightening preparation (C) containing at least one bleaching strength intensifier,
at least one preparation (Al) which contains in a cosmetic carrier at least one oxidation dye precursor, and
at least one preparation (A2), wherein preparation (A2) contains in a cosmetic carrier at least one 3,4-dihydroisoquinolinium derivative according to formula (I).

The multicomponent kit of parts preferably also contains instructions for use. It can moreover be preferable if an application aid, such as comb or a brush, and/or personal protection equipment, such as disposable gloves, are also included with the kit. All that has been stated regarding the agents applies with necessary alterations to further preferred embodiments of the multicomponent kit of parts.

Mixing of preparations (A), or (A1) and (A2), and (B) or optionally of preparations (A), or (A1) and (A2), and (B) and (C) prior to application results in an application mixture according to the invention. The actual hair coloring agent is conveniently prepared immediately before application by mixing preparations (A), or (A1) and (A2), with (B) and optionally (C). The application temperatures can be in a range from 15 to 40° C. After a contact time of 5 to 45 minutes, the hair coloring agent is removed by rinsing the hair. There is no need to wash with a shampoo afterwards if a highly surfactant-containing carrier (e.g., a coloring shampoo) is used.

The present invention therefore also provides a method for dyeing human hair wherein an agent according to the invention is applied to the hair in accordance with the above instructions, left on the hair for a contact period of 2 to 45 minutes, preferably 5 to 15 minutes, and rinsed out of the hair or washed out with a shampoo. Aplication temperatures for the color-changing and/or permanent shape-changing according to the invention of keratin fibers can be in a range from 15 to 45° C. After a contact period, the hair coloring agent is removed from the hair by rinsing. There is no need to wash with a shampoo afterwards if a highly surfactant-containing carrier (e.g., a coloring shampoo) was used.

The invention also provides for use of an agent according to the invention for dyeing and simultaneously bleaching keratinous fibers, in particular human hair. All that has been stated in respect of the agents according to the invention applies with necessary alterations to further embodiments of the methods and uses according to the invention.

The examples below are intended to illustrate preferred embodiments of the invention without however limiting it.

### EXAMPLES

### 1. Synthesis Examples

### Synthesis of N-(2-phenylethyl)formamide

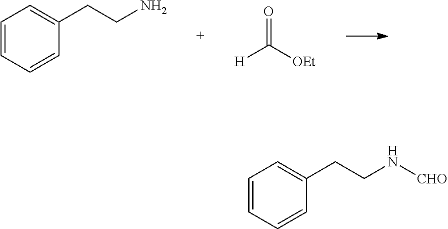

100.0 g (0.83 mol) of phenylethylamine and 187.0 g (2.07 mol) of ethyl formate were refluxed together for 12 hours. Excess ethyl formate was removed under vacuum in a rotary evaporator. A virtually colorless oil remained as residue, which was used in the next stage with no further purification. Yield: 122.2 g (99%); ¹H-NMR (400 MHz, DMSO-d₆): δ [ppm]=2.72 (t, 2H); 3.45 (t, 2H); 7.19-7.31 (m, 5H); 8.00 (s, 1H); 8.10 (br, NH); ¹³C-NMR (400 MHz, DMSO-d₆): δ [ppm]=35.0; 38.7; 125.9; 128.0; 128.2; 139.1; 160.1.

### 1.2 Synthesis of 3,4-dihydroisoquinoline

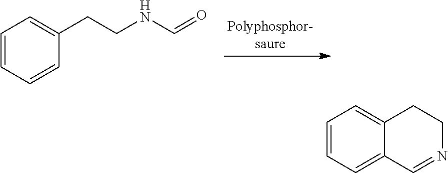

490.0 g (5.00 mol) of polyphosphoric acid were heated to 80° C. until it could be thoroughly mixed with a metal stirrer. Then 84.0 g (0.56 mol) of N-(2-phenylethyl)formamide from stage 1 was added at 80° C. while stirring and the mixture was heated to 160° C. for 12 hours. After the reaction the mixture was poured onto 1000 ml of iced water and then stirred for 2 hours at room temperature. A pH of 12.0 was established with a 5-molar, aqueous sodium hydroxide solution. The aqueous phase was extracted with methyl t-butyl ether. The combined organic phases were dried with magnesium sulfate and completely evaporated in a rotary evaporator, resulting in dark brown oil. The oil was distilled under vacuum (40 mbar/115° C.) and accumulated in the form of a clear, light-brown liquid, which was used in the third stage. Yield: 56.3 g (76%); ¹H-NMR (400 MHz, DMSO-d₆): δ [ppm]=2.68 (t, 2H); 3.65 (t, 2H); 7.23 (d, 1H); 7.34 (m, 2H); 7.41 (d, 1H); 8.34 (s, 1H); ¹³C-NMR (400 MHz, DMSO-d₆): δ [ppm]=25.4; 48.0; 127.5; 127.8; 128.9; 131.5; 137.0; 160.7.

### 1.3 Synthesis of N-methyl-3,4-dihydroisoquinolinium-p-toluenesulfonate (A1)

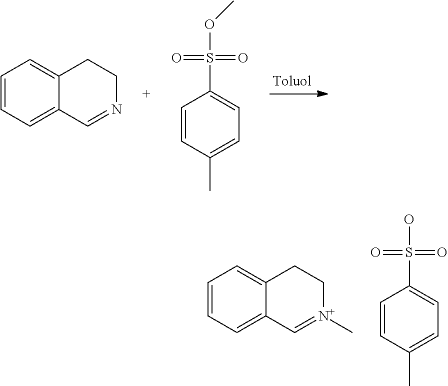

56.0 g (0.43 mol) of 3,4-dihydroisoquinoline from stage 2 were added to a solution of 80.0 g (0.43 mol) of p-toluenesulfonic acid methyl ester in 250 ml of toluene. The reaction mixture was stirred for three hours at 60° C., during which time the solution gradually became turbid. The solid which precipitated out after cooling was filtered off and dried under vacuum. Yield: 125.6 g (93%); ¹H-NMR (400 MHz, DMSO-d₆): δ [ppm]=2.25 (s, 3H); 3.18 (t, 2H); 3.72 (s, 3H); 4.01 (t, 2H); 7.09 (d, 2H); 7.20 (m, 2H); 7.52 (d, 2H); 7.58 (m, 1H); 7.79 (m, 1H); 9.23 (s, 1H);¹³C-NMR (400 MHz, DMSO-d₆): δ [ppm]=24.2; 26.8; 50.0; 52.7; 126.0; 127.3; 130.6; 130.8; 132.3; 136.0; 139.1; 140.1; 140.7; 148.1; 169.1.

### 2. Lightening Examples

### 2.1 Production of Lightening Creams

Lightening creams were produced from the constituents listed below:

| | wt. % | | |
|---|---|---|---|
| Raw material | C1 | E1 | E2 |
| Hydrenol ® D | 4.0 | 4.0 | 4.0 |
| Lorol ® C12-18, tech. | 2.4 | 2.4 | 2.4 |
| Eumulgin ® B3 | 0.2 | 0.2 | 0.2 |
| Eumulgin ® B2 | 0.2 | 0.2 | 0.2 |
| Mergital ® CS 50 A | 0.8 | 0.8 | 0.8 |
| 1,2-Propanediol | 0.4 | 0.4 | 0.4 |
| Xanthan FN | 0.1 | 0.1 | 0.1 |
| Crodafos ® CES | 2.0 | 2.0 | 2.0 |
| Amphoterge ® K-2 | 2.0 | 2.0 | 2.0 |
| Merquat ® 281 | 3.0 | 3.0 | 3.0 |
| Ammonium sulfate | 2.0 | 2.0 | 2.0 |
| Sodium sulfite | 0.1 | 0.1 | 0.1 |
| L-Arginine | 1.0 | 1.0 | 1.0 |
| p-Toluylene diamine sulfate | 0.1 | 0.1 | 0.1 |
| Resorcinol | 0.1 | 0.1 | 0.1 |
| HEDP 60% | 0.2 | 0.2 | 0.2 |
| Sodium silicate 40/42 | 0.5 | 0.5 | 0.5 |
| Ammonia 25% | 10.0 | 10.0 | 10.0 |
| Triethanolamine | — | — | 1.0 |
| N-Methyl-3,4-dihydroisoquinolinium-p- | — | 1.0 | 1.0 |
| toluenesulfonate (A1) | | | |
| Water | to 100 | to 100 | to 100 |

Raw materials used:
Hydrenol ® D INCI name: Cetearyl Alcohol (Cognis)
Lorol ® C12-18, tech. INCI name: Coconut Alcohol (Cognis)
Eumulgin ® B3 INCI name: Ceteareth-30 (Cognis)
Eumulgin ® B2 INCI name: Ceteareth-20 (Cognis)
Mergital ® CS 50 INCI name: Ceteareth-50 (Cognis)
Crodafos ® CES INCI name: Cetearyl Alcohol, Dicetyl Phosphate, Ceteth-10 Phosphate (Croda)
Amphoterge ® K-2 INCI name: Disodium Cocoamphodipropionate (Lonza)
Merquat ® 281 INCI name: Polyquaternium-22 (Nalco)

### 2.2 Mixing with the Developer Dispersion

Each lightening cream was mixed in a ratio of 1:1 with a developer dispersion having a composition as follows. The pH of the final application mixture was from 9 to 10.2.

| Raw material | wt. % |
|---|---|
| Sodium hydroxide solution 45% | 0.73 |
| Dipicolinic acid | 0.10 |
| Disodium pyrophosphate | 0.03 |
| HEDP 60% | 1.5 |
| FAEOS-Na C12-14 2EO 27% | 2.0 |
| Dow Corning ® DB 110 A | 0.07 |
| Aculyn ® 33A | 2.5 |
| Hydrogen peroxide 50% | 20.00 |
| Water | to 100 |

Dow Corning ® DB 110 A INCI name: Dimethicone (Dow Corning)
Aculyn ® 33A INCI name: Acrylates Copolymer (ISP)

For the coloring process 4 times the amount of the final application mixture was applied to strands of dark blonde hair (code Kerling 7/0) and light brown hair (code Fischbach & Miller 6923) weighing approx. 0.7 g. After the strands had been lightened for 10 minutes at 32° C. they were washed with a commercial shampoo and dried with a hairdryer.

### 2.3 Evaluation of the Lightening and Coloring Capacity

Each hair strand was measured by colorimetry before and after the bleaching process. For each measurement an L value, an a value and a b value were determined. The L value represents the lightness of the color (black-white axis); the higher the L value, the lighter the color. The a value represents the red-green axis of the system; the higher the value, the greater the shift to the red. The b value represents the yellow-blue axis of the system; the higher the value, the greater the shift to the yellow.

The dL value (change in lightening) calculated using the following equation (I) was used as a measure of the lightening capacity of each formulation:

ΔL=L_{after}−L_{before} Equation (I)

L_{after}=brightness of the strands after coloring

L_{before}=brightness of the strands before coloring

Color differences were characterized by the ΔE value, which is calculated from the three measured values in accordance with equation (II):

ΔE=\sqrt{(ΔL)²+(Δa)²+(Δb)²} Equation (II)
Δa=a_{after}−a_{before} where a_{after}=a value after coloring;
   a_{before}=a value before coloring;
Δb−b_{after}−b_{before} where b_{after}=b value after coloring;
   b_{before}=b value before coloring.

Two measurements were performed for each formulation and each hair type, and the average was calculated from the individual values in each case. The higher the ΔL value, the better the lightening capacity of the individual formulation. The higher the ΔE value, the greater the color difference in comparison to the original hair color.

### 2.4 Results of the Lightening and Coloring Capacity

Lightening and coloring capacity on
dark blonde strands (Kerling 7/0)

| ΔL (formulation C1) | ΔL (formulation E1) | ΔL (formulation E2) |
|---|---|---|
| 3.82 | 7.42 | 5.67 |
| ΔE (formulation C1) | ΔE (formulation E1) | ΔE (formulation E2) |
| 5.80 | 11.01 | 9.02 |

Lightening and coloring capacity on light
brown strands (Fischbach & Miller 6923)

| ΔL (formulation C1) | ΔL (formulation E1) | ΔL (formulation E2) |
|---|---|---|
| 4.44 | 5.12 | 6.51 |
| ΔE (formulation C1) | ΔE (formulation E1) | ΔE (formulation E2) |
| 7.38 | 9.09 | 10.18 |

A comparison of the colorimetry values clearly shows that higher ΔL values can be achieved with the formulations according to the invention. Through the use of these formulations the lightening capacities are thus improved, in that they can be classed as superior in relation to the prior art. Likewise, higher ΔE values are obtained with the formulations according to the invention than with the comparison formulations. A greater color difference can thus be achieved with the composition described in this invention—with an application time of only 10 minutes and without increasing the concentration of the formulation constituents not according to the invention that are used—in relation to the original hair color.

We claim:

1. Agent for dyeing and simultaneously lightening keratin fibers, wherein it contains in a cosmetic carrier at least one oxidation dye precursor and at least one cationic 3,4-dihydroisoquinolinium derivative according to the following formula (I) 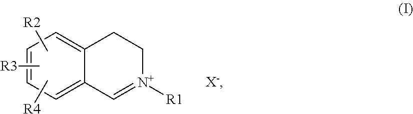
wherein R1 is a C₁-C₆ alkyl group, a C₂-C₆ alkenyl group, a C₂-C₆ hydroxyalkyl group, a C₁-C₆ alkoxy C₂-C₆ alkyl group, a carboxy C₁-C₆ alkyl group, an aryl C₁-C₆ alkyl group, a di(C₁-C₆ alkyl)amino C₂-C₆ alkyl group, a heteroaryl C₁-C₆ alkyl group, a 3-oxobutyl group, a 2-oxopropyl group, an aryl group or a heteroaryl group; R2, R3 and R4 are, independently of one another, a hydrogen atom, a hydroxyl group, an amino group, a di(C₁-C₆ alkyl)amino group, a C₁-C₆ alkyl group, a C₁-C₆ alkoxy group, halogen, a nitro group, a carboxy group, a nitrile group, an optionally substituted aryl group, a C₂-C₆ alkenyl group, an optionally substituted heteroaryl group or R2 and R3 together can form a further fused carbocyclic or heterocyclic ring, which can be saturated or unsaturated and can optionally be substituted by up to three substituents; and X⁻ is a physiologically tolerable anion.

2. Agent according to claim 1, wherein R1 is a C₁-C₆ alkyl group, a C₂-C₆ alkenyl group or a C₂-C₆ hydroxyalkyl group.

3. Agent according to claim 1, wherein R2, R3 and R4 are hydrogen.

4. Agent according to claim 1, wherein X⁻ is a halide ion, hydrogen sulfate, ½ sulfate, p-toluenesulfonate, benzenesulfonate, or acetate.

5. Agent according to claim 1, wherein the least one cationic derivative according to formula (I) is chosen from N-methyl-3,4-dihydroisoquinolinium-p-toluenesulfonate, N-methyl-3,4-dihydroisoquinolinium benzenesulfonate, N-methyl-3,4-dihydroisoquinolinium bromide, N-methyl-3,4-dihydroisoquinolinium hydrogen sulfate, N-methyl-3,4-dihydroisoquinolinium acetate, N-allyl-3,4-dihydroisoquinolinium-p-toluenesulfonate, N-allyl-3,4-dihydroisoquinolinium benzenesulfonate, N-allyl-3,4-dihydroisoquinolinium bromide, N-allyl-3,4-dihydroisoquinolinium hydrogen sulfate, N-allyl-3,4-dihydroisoquinolinium acetate, 3,4-dihydro-2-(3-hydroxypropyl)isoquinolinium-p-toluenesulfonate, 3,4-dihydro-2-(3-hydroxypropyl)isoquinolinium benzenesulfonate, 3,4-dihydro-2-(3-hydroxypropyl)isoquinolinium bromide, 3,4-dihydro-2-(3-hydroxypropyl)isoquinolinium hydrogen sulfate, 3,4-dihydro-2-(3-hydroxypropyl)isoquinolinium acetate, 3,4-dihydro-2-(2-hydroxyethyl)isoquinolinium-p-toluenesulfonate, 3,4-dihydro-2-(2-hydroxyethyl)isoquinolinium benzenesulfonate, 3,4-dihydro-2-(2-hydroxyethyl)isoquinolinium bromide, 3,4-dihydro-2-(2-hydroxyethyl)isoquinolinium hydrogen sulfate or 3,4-dihydro-2-(2-hydroxyethyl)isoquinolinium acetate.

6. Agent according to claim 1, wherein the at least one cationic 3,4-dihydroisoquinolinium derivative according to formula (I) is present in an amount from 0.01 to 15 wt. %, based on total weight of the ready-to-use agent.

7. Agent according to claim 1, wherein it is prepared directly before use by mixing at least two preparations, the at least two preparations being provided in at least two separately packaged containers, with one container containing a dye (A) having in a cosmetic carrier at least one oxidation dye precursor and at least one 3,4-dihydroisoquinolinium derivative according to formula (I), and a second container containing an oxidizing agent preparation (B) containing at least one oxidizing agent.

8. Agent according to claim 7, wherein the oxidizing agent of preparation (B) is hydrogen peroxide and/or one of its solid addition products with organic or inorganic compounds.

9. Agent according to claim 8, wherein the oxidizing agent is present in an amount of 0.5 to 12 wt. % (calculated as H₂O₂100%), based on total weight of the agent.

10. Agent according to claim 1, wherein the pH of the agent is from 7 to 11.

11. Agent according to claim 1 further comprising triethanolamine.

12. Method for lightening keratin fibers comprising applying an agent according to claim 1 to the keratinous fibers, leaving the agent on the fibers for 5 to 45 minutes, and rinsing out the agent with water or washing out the agent with a shampoo.
